(19) United States
(12) Patent Application Publication
(10) Pub. No.: US 2022/0242808 A1
(43) Pub. Date: Aug. 4, 2022

(54) PROCESS FOR PREPARING ALKANEDIOLS

(71) Applicant: SYMRISE AG, Holzminden (DE)

(72) Inventors: Bernd HÖLSCHER, Halle (DE); Marc MANSFELD, Brevörde (DE)

(21) Appl. No.: 17/281,097

(22) PCT Filed: Oct. 2, 2018
(86) PCT No.: PCT/EP2018/076862

§ 371 Date: Oct. 21, 2021

(51) Int. Cl. C07C 29/44

(52) U.S. Cl. CPC C07C 29/44

(57) ABSTRACT

A process is proposed for the production of longer-chain 1,2-alkanediols and 1,3-alkanediols by a radical-chain addition reaction of alkylene glycols to obtain olefins.

### FIELD OF THE INVENTION

The invention is in the field of cosmetic raw materials and relates to a process for the production of longer-chain alkanediols by means of a radical-chain addition reaction.

### BACKGROUND TECHNOLOGY

Alkanediols, specifically 1,2-alkanediols and 1,3-alkanediols, are important additives in the cosmetic industry, serving as starting materials for the synthesis of acetals which are used in the fragrance industry.

The most important alkanediols include 1,2-hexanediol, 1,2-octanediol, 1,2-decanediol, and particularly 1,2-pentanediol. However, a significant disadvantage is in that the products always exhibit an unpleasant odour which needs to be masked with considerable effort, particularly in cosmetic final formulations. In addition, conventional production thereof is technically complex, requiring a high input of water which subsequently needs to be purified with considerable effort.

### RELEVANT STATE OF THE ART

For example, the production of 1,2-pentanediol is based on furfuryl alcohol which is obtained in large amounts as a waste product of the production of sugar from cereal grains.

For example, Adkins and Connor [Journal of American Chemical Society 53, 1091 (1931)] report that the hydration or hydrogenolysis of furfuryl alcohol at 175° C. in a liquid phase using copper chromite as a catalyst yields a mixture of 40% 1,2-pentanediol, 30% 1,5-pentanediol, 10% amyl alcohol, and 20% tetrahydrofurfuryl alcohol and methyltetrahydrofuran.

Kaufmann and Adams [Journal of American Chemical Society 45, 3029 (1923)] describe that the hydration of furfural in the presence of platinum black at room temperature yields a mixture of furfuryl alcohol, 1-pentanol, tetrahydrofurfuryl alcohol, 2-pentanediol, and 1,5-pentanediol.

Furthermore, the paper by Smith and Fuzek [Journal of American Chemical Society 71, 415 (1949)] describes studies on the catalytic hydration or hydrogenolysis of furan and furan derivatives in the liquid phase by means of platinum dioxide catalysts. The reactions were performed in acetic acid at a hydrogen pressure of 20, 40, or 60 psi, the catalyst mentioned was prepared according to instructions [Organic Synthesis 8, 92 (1928)]. During the hydration or hydrogenolysis of furfuryl alcohol using platinum dioxide as a catalyst, 1,2-pentanediol is purportedly obtained in nearly the same quantitative yield; 1,2-pentanediol was separated from the acetic acid in the form of the diacetate.

A hydration of furfuryl alcohol to obtain 1,2-pentanediol in the presence of heterogeneous catalysts is also described in WO 2012 152 849 A1 (SYMRISE).

EP 1876162 A1 describes the production of alkanediols from the respective olefins by means of epoxidation and subsequent hydrolysis. The raw materials such obtained were further purified by a post-treatment in order to remove byproducts which have an unpleasant odour. According to U.S. Pat. No. 6,528,665 B1, purification is performed at the step of the epoxy-alkanes before hydrolysing them to obtain the corresponding alkanediols.

Nowadays, the production of 1,2-pentanediol is generally based on n-pent-1-ene which is available from petrochemical sources. n-pent-1-ene is reacted by means of peroxides (e.g., hydrogen peroxide) to obtain the corresponding epoxide, and is then reacted with organic acids, such as formic acid or mineral acids, to obtain 1,2-pentanediol (cf. EP 0257243 A1 or EP 0141775 A1).

### OBJECT OF THE INVENTION

Therefore, a first object of the present invention was to provide an alternative process for the production of longer-chain alkanediols, specifically of 1,2-alkanediols and 1,3-alkanediols containing 5 to 12 carbon atoms, which do not exhibit the disadvantages initially described. The products should be odour-free, and the process should possibly be characterised by a simple design and a low amount of waste products obtained. Further, it should also be possible to perform this process using renewable resources. This is ensured when the olefins are produced from native alcohols while the diols origin from natural resources.

### DESCRIPTION OF THE INVENTION

A first subject matter of the invention relates to a process for the production of alkanediols of the formula (I)

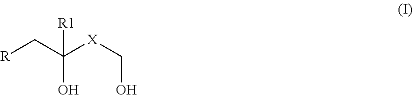

in which R is a linear or branched alkyl group containing 1 to 12 carbon atoms, R¹ is hydrogen or a methyl group, and X is zero or a CH₂ group, consisting of or comprising the following steps:
(a) providing a mixture containing at least one olefin and at least one alkylene glycol as well as, optionally, at least one solvent;
(b) adding at least one radical initiator to the mixture of step (a);
(c) heating the mixture of step (b) to about 100 to 200° C.;
(d) separating the unreacted reactants and, optionally, the solvent from the alkanediols obtained; and, optionally,
(e) purifying the alkanediols.

A second subject matter of the invention relates to a process for the production of alkanediols, which is a modified version compared to of the first alternative, of the formula (I)

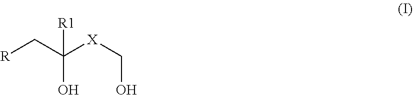

in which R is a linear or branched alkyl group containing 1 to 12 carbon atoms, R¹ is hydrogen or a methyl group, and X is zero or a CH₂ group, consisting of or comprising the following steps:
(a) providing a first mixture containing at least one alkylene glycol and at least one solvent;
(b) providing a second mixture containing at least one olefin, at least one radical initiator and at least one solvent;
(c) heating the first mixture to about 100 to 200° C.;
(d) adding small amounts of the second mixture to the heated first mixture;
(e) separating the unreacted reactants and the solvents from the alkanediols obtained; and, optionally,
(e) purifying the alkanediols.

Surprisingly, it was found that olefins can be reacted with alkylene glycols during a radical-chain addition reaction wherein 1,2-alkanedios or 1,3-alkanediols of formulae (la) and (b) are obtained,

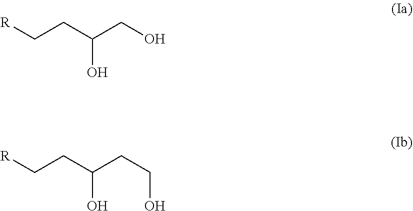

in which R is a linear or branched alkyl group containing 1 to 12 carbon atoms. The radical-chain addition reaction proceeds in short times and leads to high yields. The products are odour-free, the technical effort is low and there are practically no side products or byproducts which need to be discharged at an effort.

### Olefins

The selection of olefins depends on the consideration of what chain length the desired alkanediol should have. If a 1,2-alkanediol is desired, the final chain length is deter-mined by the chain length of the olefin plus 2, if a 1,3-alkanediol is desired, it is the chain length of the olefin plus 3. Suitable for this purpose are propene, butene, pentene, hexene, heptene, octene, nonene, decene, undecene, dodecene, and mixtures thereof. Preferably, these are terminal olefins, however, internal olefins may also be used. Because the olefins themselves can be obtained from renewable resources, specifically alcohols, it is possible that the alkanediols of the present invention may also be obtained without using any petrochemical starting products at all.

### Alkylene Glycols

If 1,2-alkanediols are to be obtained, ethylene glycol is used as alkylene glycol. If 1,3-alkanediols are desired, 1,3-propylene glycol is used. Mixtures may of course also be used. Usually, the olefins and the alkylene glycols are used in a ratio of the equivalents of about 1:1 to about 1:50, preferably of about 1:10 to about 1:40, and more preferably of about 1:20 to about 1:30.

### Radical Initiators

The process of the invention is a radical-chain addition reaction which proceeds as follows:
Radikalstarter:
organische Peroxide, Metalloxide and Licht

A-B→A.+B.
Reaktionsmechanismus
derRadikaladdition

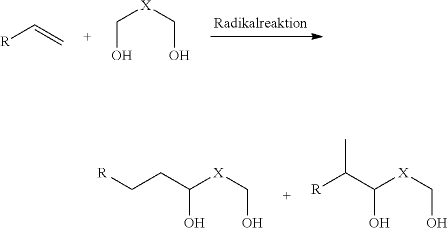

[Radical starters:
organic peroxides, metal oxides, and light
Reaction mechanism of the radical-chain addition reaction; radical reaction]

Radicals are required to start the radical-chain addition reaction. Therefore, radical initiators are added to the mixture, which may be, for example, peroxides, such as, for example, tert-butyl peroxide or benzoyl peroxide; further examples can be found in the publications DE 2136496 A1 and DE 19853862 A1. Alternatively, azo compounds, such as azoisobutyronitrile, may also be employed.

Instead of peroxides, metal oxides are suitable, specifically metal oxides of transition metals, those being, in particular, copper oxides, ferric oxides, manganese oxides, indium oxides, cobalt oxides, silver oxides, and mixtures thereof. Particularly suitable for performing this process are metal oxides, selected from Ag₂O, CuO, Fe₂O₃, Fe₃O₄, CuFe₂O₄, Co₃O₄, CoO, MnO₂, In₂O₃, and mixtures thereof. Preferably, the metal oxides may be employed as powders or granules. The metal oxides may also be applied onto a suitable inorganic carrier material, such as, for example, aluminium oxide.

Usually, the radical initiators are employed in amounts of about 0.5 to about 2% by weight, and particularly of about 1 to about 1.5% by weight, based on the total amounts of olefins and alkylene glycols.

Finally, it may be advantageous to initiate the radical reaction by means of a light-induced manner, i.e., performing the reaction while exposed to light of a suitable wave-length.

### Solvents

In a preferred embodiment, the radical-chain addition reaction is performed within a solubilising agent. To this end, particular nonpolar solvents are suitable which are not able to form radicals themselves, or are able to do so with difficulty, such as ether (e.g., methyl tert-butyl ether) and particular dialkyl carbonates, such as dimethyl carbonate or diethyl carbonate. Those are separated by distillation after completion of the reaction and may be re-turned to the process.

### Reaction Conditions and Purification

The reaction is performed at an increased temperature. Suitable ranges of temperature are about 100 to about 200° C. Particularly preferred are temperatures within the range of about 150 to about 180° C.

It has proven to be advantageous to perform the reaction under an inert gas, specifically under a nitrogen atmosphere. The latter may initially have a pressure of 1 to 5 bar, which will increase correspondingly under reaction conditions. Then, the reaction proceeds under autogenous pressure which may amount to, for example, 10 to 20 bar.

The two processes claimed may be performed continuously or discontinuously. For example, a discontinuous stirred tank reactor is suitable for performing the discontinuous process. For the continuous process, for example, a continuous tube reactor, stirred tank reactor, fixed-bed reactor, or trickle-bed reactor is suitable.

After performing the process, there is a product mixture which is a mixture of varying concentrations of educts and products depending on the duration of the process and the process parameters used. The product mixture can be separated by suitable separation processes, particularly by means of distillation, which allows to further increase the high purity of the desired alpha-monoalkyl products, and, optionally, to recover and re-employ unreacted educts. Preferably, purification is performed in a Spaltrohr® column.

### INDUSTRIAL APPLICABILITY

The alkanediols obtained according to the process of the invention are suitable as additives for the cosmetic industry, but also for detergents and cleaning agents. After acetalisation, they may also be employed as fragrants or precursors thereof.

### EXAMPLES

### General Process of Production

A 50% by weight solution/mixture within dimethyl carbonate of 1 equivalent olefin, 30 equivalents alkylene glycol and 1% by weight, based on the sum of olefin and glycol, di-tert-butylperoxide is placed in an autoclave and heated up to 155 to 160° C. at an initial nitrogen pressure of 5 bar. The pressure will increase to 15-20 bar. The mixture is stirred at this temperature for 1 hour and is then allowed to cool down to room temperature. Dimethyl carbonate and excess glycol are distilled off under vacuum conditions. The residue obtained in a 30 cm column is distilled under vacuum conditions.

### Example 1

### Production of 1,2-decanediol

149 g (1.33 mol) 1-octene, 816 g (13.16 mol) 1,2-ethylene glycol, 975 g dimethyl carbonate and 19.4 g (0.13 mol) di-tert-butylperoxide were placed into a 5 litre autoclave and heated while stirring at an initial nitrogen pressure of 5 bar. The mixture was stirred for 1 h at 155-160° C. In doing so, the pressure increased to 11 bar. The mixture was cooled down and distilled.

Fraction 1 (rotation evaporator: bath: 60-80° C., head: 43-48° C., vacuum: 170-60 mbar)=983 g, 90% dimethyl carbonate after GC,

Fraction 2 (10 cm Vigreux column with water bath: bath: 73-90° C., head: 63-65° C., vacuum: 0.5 mbar): 797 g, 95% 1,2-ethylene glycol according to GC,

Fraction 3 (10 cm Vigreux column with multi-limb vacuum receiver adapter: sump: 135-175° C., head: 116-135° C., vacuum: 0.5 mbar): 17.3 g 1,2-decanediol, 97% according to GC.

### General Process of Production Including Dosage

A 1:1 mixture of alkylene glycol (40 equivalents, based on olefin) and dimethyl carbonate is placed into an autoclave and heated to 155-160° C. at an initial nitrogen pressure of 5 bar. At this temperature, a ca. 75% by weight solution in dimethyl carbonate made of 1 equivalent olefin, 0.35 equivalents di-tert-butylperoxide is added in small amounts in the course of 1.5 hours. Pressure will increase to 15-20 bar. The mixture is stirred for another 15 min under these conditions and then allowed to cool down to room temperature. Dimethyl carbonate and excess diol are distilled off. A purifying distillation of the raw product in a Spaltrohr® column (Fischer Spaltrohr® column HMS 500 AC) is performed. The yield will amount to 35-50% of the theory (based on the olefin used).

### Example 2

### Preparation of 1,2-decanediol

1.200 g (19.4 mol) 1,2-ethylene glycol and 900 g dimethyl carbonate were placed into a 5 litre autoclave and heated to a temperature of 155 to 160° C. at an initial pressure of 5 bar. At this temperature, a solution of 54 g (0.48 mol) 1-octene, 210 g dimethyl carbonate and 24.6 g (0.17 mol) di-tert-butylperoxide was added in small amounts within the course of 1.5 hours. Subsequently, the mixture was stirred for another 15 minutes and was then allowed to cool down to room temperature. Dimethyl carbonate and excess 1,2-ethylene glycol were distilled off in the rotary evaporator. There remained a residue of 62.1 g 1,2-decanediol with a purity of 52% according to GC. The raw product was distilled in a 30 cm column. Subsequently, the yield amounted to 34.6 g 1,2-decanediol with a purity of 98% according to GC.

### Examples 3 to 23

The following 1,2-alkanediols were prepared in the same manner. Table 1 shows the structures, molar masses, and data from mass spectroscopy.

TABLE 1
1,2-alkanediols

| Structure | MG | MS |
|---|---|---|
| 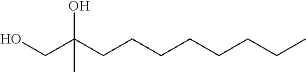 | 188 | m/z: 43 (59), 55 (45), 57 (52), 69 (40), 71 (29), 75 (100), 83 (62), 97 (32), 157 (70), |
| | | 173 (3) |
| 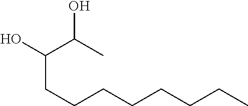 | 188 | m/z: 43 (25), 55 (40), 57 (32), 69 (100), 75 (4), 83 (48), 97 (5), 124 (3), 143 (11) |
| 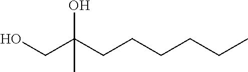 | 160 | m/z: 43 (69), 45 (27), 57 (39), 69 (100), 75 (89), 85 (8), 97 (9), 111 (5), 129 (87), |
| | | 145 (4) |
| 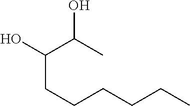 | 160 | m/z: 43 (27), 45 (22), 55 (100), 57 (14), 69 (18), 75 (5), 81 (4), 85 (1), 97 (64), 115 (15) |
| 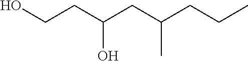 | 160 | m/z: 43 (63), 55 (39), 57 (49), 70 (56), 75 (100), 85 (9), 97 (26), |
| | | 109 (2), 113 (14) |
| 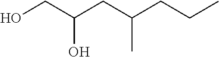 | 146 | m/z: 43 (82), 55 (61), 57 (10), 61 (14), 69 (10), 71 (47), 81 (2), 85 |
| | | (2), 97 (100), 115 |
| | | (32) |
| 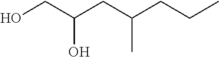 | 146 | m/z: 43 (82), 55 (63), 57 (10), 61 (16), 69 (15), 71 (47), 81 (2), 85 |
| | | (2), 97 (100), 115 |
| | | (33) |
| 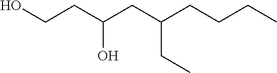 | 188 | m/z: 41 (30), 57 (100), 70 (47), 75 (77), 83 (19), 98 (48), 112 (8), 123 (4), 143 (6), 168 |
| | | (1) |
| 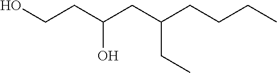 | 188 | m/z: 41 (25), 57 (100), 69 (33), 75 (75), 83 (16), 98 (52), 113 (5), 123 (4), 143 (7) |
| 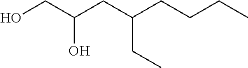 | 174 | m/z: 43 (20), 57 (100), 61 (6), 69 (41), 83 (22), 98 (6), 109 (2), 125 (4), 143 (18) |
| 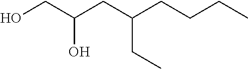 | 174 | m/z: 43 (20), 57 (100), 61 (7), 69 (42), 83 (23), 98 (6), 109 (2), 125 (5), 143 (18) |
| 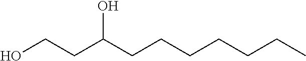 | 174 | m/z: 41 (26), 55 (30), 57 (74), 69 (43), 75 (100), 84 (13), 95 (5), 100 |
| | | (4), 111 (7), 127 |
| | | (18) |
| 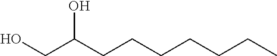 | 160 | m/z: 43 (20), 55 (35), 57 (13), 61 (10), 69 (100), 83 (5), 95 (1), 111 |
| | | (22), 129 (18) |
| 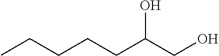 | 132 | m/z: 41 (31), 43 (25), 55 (100), 57 (13), 61 (14), 69 (2), 83 (91), |
| | | 101 (27) |
| 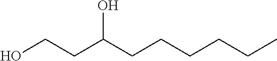 | 160 | m/z: 43 (36), 55 (53), 57 (61), 70 (22), 75 (100), 81 (11), 85 (12), |
| | | 97 (18), 113 (19) |
| 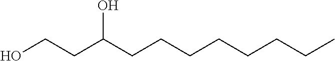 | 188 | m/z: 43 (24), 57 (56), 69 (28), 75 (100), 83 (16), 98 (7), 114 (2), |
| | | 124 (3), 141 (13) |
| 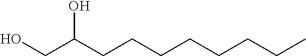 | 174 | m/z: 41 (27), 55 (42), 61 (11), 69 (100), 83 (43), 97 (5), 109 (1), 125 |
| | | (3), 143 (21) |
| 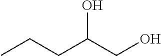 | 104 | m/z: 31 (41), 39 (15), 43 (59), 45 (10), 55 (100), 57 (9), 61 (24), |
| | | 69 (1), 73 (60) |
| 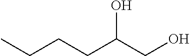 | 118 | m/z: 31 (13), 41 (48), 43 (27), 45 (11), 53 (1), 57 (12), 61 (15), 69 |
| | | (100), 82 (1), 87 |
| | | (44) |
| 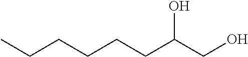 | 146 | m/z: 43 (31), 55 (100), 57 (7), 61 (11), 69 (15), 81 (2), 85 (1), 97 |
| | | (44), 115 (13) |

1. A process for the production of alkanediols of the formula (I) 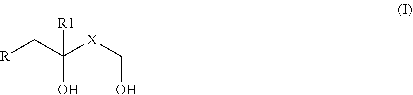
in which R is a linear or branched alkyl group containing 1 to 12 carbon atoms, R1 is hydrogen or a methyl group, and X is zero or a CH₂ group, comprising the following steps:
(a) providing a mixture containing at least one olefin and at least one alkylene glycol as well as, optionally, at least one solvent;
(b) adding at least one radical initiator to the mixture of step (a);
(c) heating the mixture of step (b) to about 100 to 200° C.;
(d) separating any unreacted reactants and, optionally, the solvent from the alkanediols obtained; and, optionally,
(e) purifying the alkanediols.

2. A process for the production of alkanediols of the formula (I) 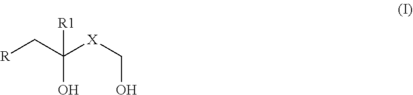
in which R is a linear or branched alkyl group containing 1 to 12 carbon atoms, R1 is hydrogen or a methyl group, and X is zero or a CH₂ group, consisting of or comprising the following steps:
(a) providing a first mixture containing at least one alkylene glycol and at least one solvent;
(b) providing a second mixture containing at least one olefin, at least one radical initiator, and at least one solvent;
(c) heating the first mixture to about 100 to 200° C.;
(d) adding small amounts of the second mixture to the heated first mixture;
(e) separating the unreacted reactants and the solvent from the alkanediols obtained; and, optionally,
(e) purifying the alkanediols.

3. The process of claim 1, wherein the 1,2-alkanediols of formula (Ia) or 1,3-alkanediols of formula (Ib) 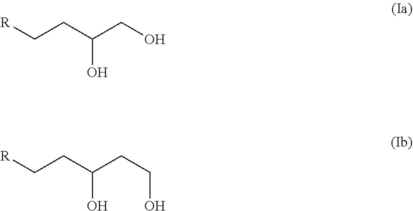
are obtained, in which R is a linear or branched alkyl group containing 1 to 12 carbon atoms.

4. The process of claim 1, wherein olefins are used, which are selected from the group consisting of propene, butene, pentene, hexene, heptene, octene, nonene, decene, undecene, dodecene, and mixtures thereof.

5. The process of claim 1, wherein ethylene glycol and/or propylene glycol are used as alkylene glycols.

6. The process of claim 1, wherein the olefins and the alkylene glycols are used in a ratio of the equivalents of about 1:1 to about 1:50.

7. The process of claim 1, wherein peroxides and/or azo compounds are used as radical initiators.

8. The process of claim 1, wherein metal oxides are used as radical initiators.

9. The process of claim 1, wherein the radical initiators are used in amounts of about 0.5 to about 2% by weight, based on the total weight of olefins and alkylene glycols.

10. The process of claim 1, wherein the reaction is performed while exposed to light.

11. The process of claim 1, wherein solvents are used which are selected from the group consisting of dialkyl ethers, dialkyl carbonates and mixtures thereof.

12. The process of claim 1, wherein the reaction is performed at a temperature within the range of about 150 to about 180° C.

13. The process of claim 1, wherein the reaction is performed under an inert gas.

14. The process of claim 1, wherein the reaction is performed under autogenous pressure.

15. The process of claim 1, wherein the alkanediols are purified in a Spaltrohr® column.

16. The process of claim 2, wherein the 1,2-alkanediols of formula (Ia) or 1,3-alkanediols of formula (Ib) 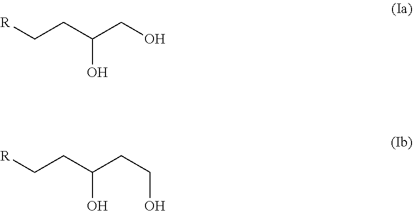
are obtained, in which R is a linear or branched alkyl group containing 1 to 12 carbon atoms.

17. The process of claim 2, wherein olefins are used, which are selected from the group consisting of propene, butene, pentene, hexene, heptene, octene, nonene, decene, undecene, dodecene, and mixtures thereof.

18. The process of claim 2, wherein ethylene glycol and/or propylene glycol are used as alkylene glycols.

19. The process of claim 2, wherein the olefins and the alkylene glycols are used in a ratio of the equivalents of about 1:1 to about 1:50.

20. The process of claim 2, wherein
peroxides and/or azo compounds are used as radical initiators, or
metal oxides are used as radical initiators, or
the radical initiators are used in amounts of about 0.5 to about 2% by weight, based on the total weight of olefins and alkylene glycols, or
the reaction is performed while exposed to light, or
solvents are used which are selected from the group consisting of dialkyl ethers, dialkyl carbonates and mixtures thereof, or
the reaction is performed at a temperature within the range of about 150 to about 180° C., or
the reaction is performed under an inert gas, or
the reaction is performed under autogenous pressure, or
the alkanediols are purified in a Spaltrohr® column.
